(19) United States
(12) Patent Application Publication
(10) Pub. No.: US 2017/0095150 A1
(43) Pub. Date: Apr. 6, 2017

(54) METHOD OF DETECTING CONSTRICTION OF PUPILS, APPARATUS, AND STORAGE MEDIUM

(71) Applicant: FUJITSU LIMITED, Kawasaki-shi (JP)

(72) Inventors: Teruyuki Sato, Tama (JP); Norihiro Kakuko, Kawasaki (JP)

(73) Assignee: FUJITSU LIMITED, Kawasaki-shi (JP)

(21) Appl. No.: 15/270,704

(22) Filed: Sep. 20, 2016

(30) Foreign Application Priority Data
Oct. 2, 2015 (JP) 2015-197117

(51) Int. Cl. A61B 3/11; A61B 3/02; A61B 3/00

(52) U.S. Cl. CPC A61B 3/112; A61B 3/0058; A61B 3/02

(57) ABSTRACT

A method of detecting constriction of the pupils includes: registering one or more motion illusion pattern images or one or more luminance values of the one or more motion illusion pattern images, the one or more motion illusion pattern images including a first motion illusion pattern image which had caused motion illusion to a specific user; displaying the registered one or more motion illusion pattern images or one or more motion illusion pattern images created based on the registered one or more luminance values on a display device; and determining whether the motion illusion is occurred to the specific user based on the displaying.

### CROSS-REFERENCE TO RELATED APPLICATION

This application is based upon and claims the benefit of priority of the prior Japanese Patent Application No. 2015-197117, filed on Oct. 2, 2015, the entire contents of which are incorporated herein by reference.

### FIELD

The embodiment discussed herein is related to a method of detecting constriction of pupils, an apparatus, and a storage medium.

### BACKGROUND

In general, when having a feeling of fatigue or drowsiness, a person has symptoms of bloodshot eyes or constriction of the pupils in which the diameters of the pupils become small as compared to a case with no feeling of fatigue or drowsiness. A known technique exploiting these symptoms detects the degree of bloodshot eyes from an image of the eyes of a person captured through a camera and detects the fatigue of this person based on the degree of bloodshot eyes. Another known technique captures an image of the pupils of a person through a camera and detects any change in the diameters of the pupils based on the captured image and detects the fatigue of this person based on the change in the diameters of the pupils.

Examples of the conventional techniques include Japanese Laid-open Patent Publication Nos. 2012-211959 and 2004-357760.

### SUMMARY

According to an aspect of the invention, a method of detecting constriction of the pupils includes: registering one or more motion illusion pattern images or one or more luminance values of the one or more motion illusion pattern images, the one or more motion illusion pattern images including a first motion illusion pattern image which had caused motion illusion to a specific user; displaying the registered one or more motion illusion pattern images or one or more motion illusion pattern images created based on the registered one or more luminance values on a display device; and determining whether the motion illusion is occurred to the specific user based on the displaying.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 illustrates an exemplary motion illusion pattern;

FIG. 2 illustrates an exemplary motion illusion pattern illustrated in FIG. 1 with an entire luminance 20% lower than that of FIG. 1;

FIG. 3 illustrates models of white, light gray, dark gray, and black included in a motion illusion pattern;

FIG. 4 is a diagram for describing a method of calculating the intensity of light arriving at a pupil from a collection of point light sources;

FIG. 5 illustrates a comparison of the intensity of light arriving at a retina;

FIG. 6 is a diagram for describing a mismatch in focusing on an object in the real world and a virtual image when a monocular HMD is used;

FIG. 7 is a diagram for describing a mismatch in the position of convergence on an object in the real world and a virtual image when a binocular HMD is used;

FIG. 8 illustrates an exemplary configuration of a pupil constriction detecting device according to the present embodiment;

FIG. 9 is a diagram for describing a luminance value in generation of a motion illusion pattern;

FIG. 10 illustrates a schematic configuration of a computer serving as the pupil constriction detecting device according to the present embodiment;

FIG. 11 is a flowchart illustrating an exemplary pre-operation processing executed at the pupil constriction detecting device according to the present embodiment;

FIG. 12 is a diagram for describing an exemplary display of a motion illusion pattern on a display;

FIG. 13 illustrates an exemplary motion illusion pattern with different kinds (shapes) of patterns from those of the motion illusion pattern illustrated in FIGS. 1 and 2;

FIG. 14 illustrates an exemplary motion illusion pattern illustrated in FIG. 13 with an entire luminance 20% lower than that of FIG. 13;

FIG. 15 is a flowchart illustrating an exemplary pupil constriction detecting processing executed at the pupil constriction detecting device according to the present embodiment; and

FIG. 16 is another flowchart illustrating an exemplary pre-operation processing executed at the pupil constriction detecting device according to the present embodiment.

### DESCRIPTION OF EMBODIMENT

When a head mounted display (HMD) is used, additional fatigue due to the configuration of the HMD is felt in addition to visual fatigue due to continuous look at a display during an operation with a visual display terminal (VDT).

For example, when a monocular HMD is used, a mismatch (difference) occurs in focusing on a virtual image displayed on the HMD and an object in the real world. In addition, a phenomenon called binocular rivalry occurs when the monocular HMD is used. When a binocular HMD is used, a mismatch (difference) occurs in the position of convergence on a virtual image displayed on the HMD and an object in the real world. These HMD specific phenomena lead to a tendency that, in general, when an HMD is used, a user is likely to feel fatigue as compared to a case in which a typical display such as an LCD or a CRT is used. For this reason, when an HMD is used, it is desirable to detect the fatigue of the user and perform, for example, restriction of continuous use to avoid excess fatigue.

The conventional technique uses an image capturing apparatus such as a camera to capture an image of the eyes (pupils) of a person, which is an increase in hardware, and thus its application to the HMD is not preferable. For example, the increase in hardware increases the size and weight of the HMD, and thus increases a load on a user on which the HMD is mounted, doubling the fatigue of the user.

Thus, a technique is desired that has a simple configuration allowing its application to the HMD and detects constriction of the pupils of a user.

It is an object of an aspect of the technique according to the present disclosure to detect constriction of the pupils of a user with a simple configuration.

Exemplary embodiments of the disclosed technique will be described below in detail with reference to the accompanying drawings.

The following first describes the principle of detection of constriction of the pupils according to the present embodiment.

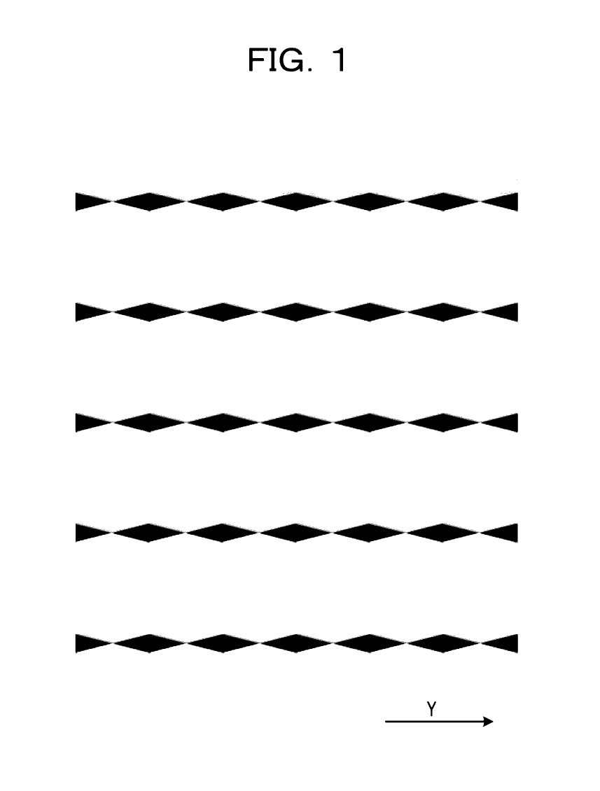

The visual perception of a human has a characteristic of suffering motion illusion. Motion illusion is a phenomenon that, when an image of an object at rest is watched, the object appears moving. A typical image that causes motion illusion is an image in which a particular luminance pattern is repeatedly arranged. Known examples of such an image include the image of a pattern called the Fraser-Wilcox illusion (hereinafter, a pattern image that causes motion illusion is referred to as a “motion illusion pattern”). The motion illusion pattern is an image in which a particular luminance pattern causes a neuron that detects motion to fire. When a human visually perceives an image in which a pattern (hereinafter referred to as a “luminance pattern”) including black, dark gray, white, light gray, and black images being repeated in a certain direction, motion illusion occurs in which the pattern appears to be moving in the direction in which the luminance pattern is repeated. FIG. 1 illustrates an exemplary motion illusion pattern. In the exemplary motion illusion pattern illustrated in FIG. 1, motion illusion occurs in which a luminance pattern appears to be moving in the direction of arrow Y in FIG. 1.

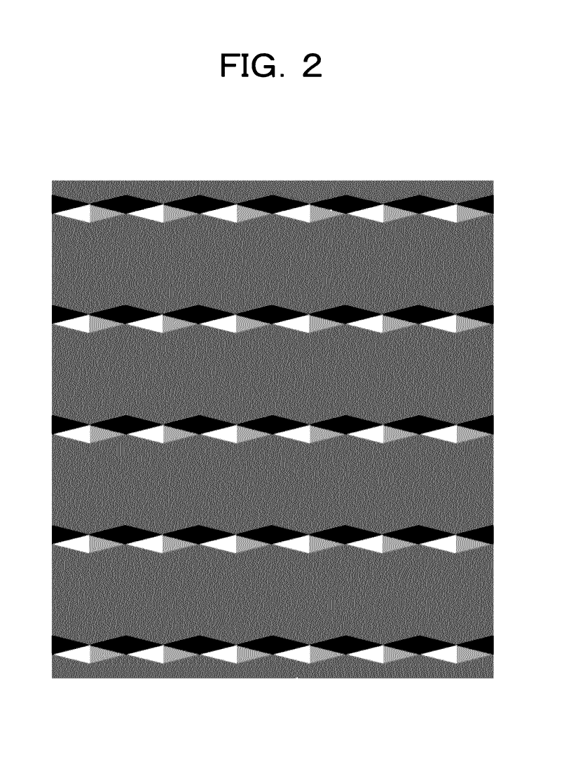

Motion illusion caused by watching an image in which a luminance pattern is repeated, in general, a larger amount of illusion is caused by a higher luminance and a higher contrast of the luminance pattern. FIG. 2 illustrates an exemplary motion illusion pattern illustrated in FIG. 1 with an entire luminance 20% lower than that of FIG. 1. Since the image illustrated in FIG. 2 has an entire brightness (luminance) 20% lower than that of the motion illusion pattern illustrated in FIG. 1, the motion illusion pattern illustrated in FIG. 2 is less likely to cause motion illusion than the motion illusion pattern illustrated in FIG. 1.

Whether an identical motion illusion pattern causes motion illusion depends on individual persons. When constriction of the pupils has occurred to an identical person, the amount of illusion by a motion illusion pattern that has caused motion illusion becomes small or motion illusion disappears.

Generally, the diameters of the pupils of human eyes are adjusted depending on a balance between the sympathetic nerve and the parasympathetic nerve, which is the function of the autonomic nervous system. For example, when a person is excited or surprised, the sympathetic nerve is dominant over the parasympathetic nerve, and thus the pupils of the person dilate. In contrast, when a person is feeling fatigue or drowsiness, the parasympathetic nerve is dominant over the sympathetic nerve, and thus the pupils of the person constrict.

A relative brightness between regions having different luminance within an image does not change at the constriction of the pupils. In contrast, when the constriction of the pupils occurs, the intensity of light arriving at a pupil (retina) decreases in proportion to the square of the radius of the pupil. Thus, the constriction of the pupils causes a phenomenon that an image is visually recognized at a compressed contrast. The following describes this phenomenon.

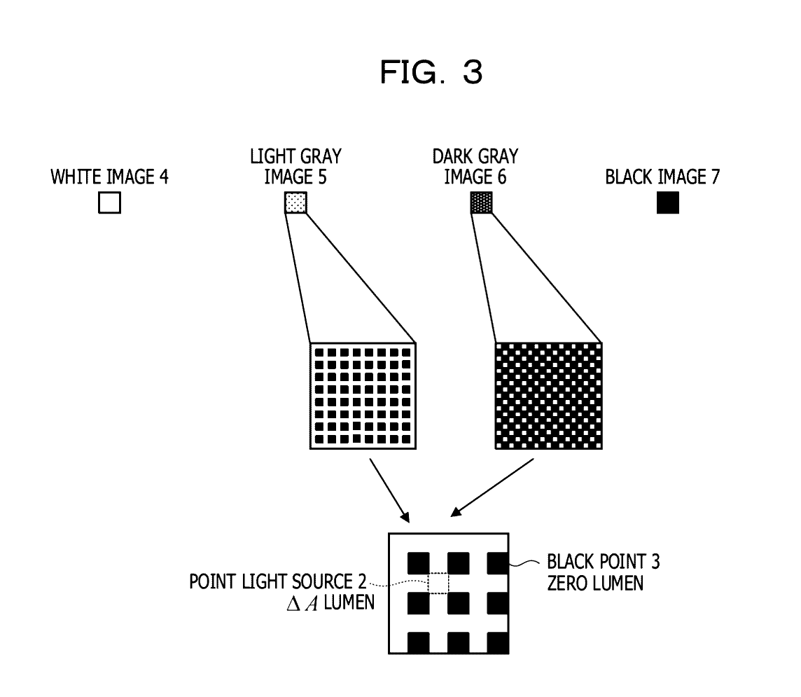

FIG. 3 illustrates models of white, light gray, dark gray, and black included in a motion illusion pattern. For example, white, light gray, dark gray, and black included in the exemplary motion illusion pattern illustrated in FIG. 1 are each modeled as a collection of minute point light sources 2 as illustrated in FIG. 3.

As illustrated in FIG. 3, a light gray image 5 and a dark gray image 6 each includes a minute point light source 2 having the intensity of light per unit angle at ΔA lumen (candela/steradian) and a minute black point 3 as a light source having the intensity of light at zero lumen, and the number of point light sources 2 becomes large as compared to the number of black points 3 as gray becomes darker. Hereinafter, the fraction of the point light sources 2 in the whole image is referred to as a “minute light source ratio k”. A white image 4 includes the point light sources 2 only and has the minute light source ratio k at one (k=1). A black image 7 includes the black points 3 only and has the minute light source ratio k at zero (k=0).

The intensity of light when the white image 4 is regarded as a light source is given by Formula (1).

ΣΔA=A[Im](k=1) (1)

The intensity of light when the light gray image 5 is regarded as a light source is given by Formula (2).

ΣΔA=k₁A[Im](k=k₁,0<k₁<1) (2)

The intensity of light when the dark gray image 6 is regarded as a light source is given by Formula (3).

ΣΔA=k₂A[Im](k=k₂,0<k₂<k₁<1) (3)

The intensity of light when the black image 7 is regarded as a light source is given by Formula (4).

ΣΔA=Σ0=zero lumen(k=0) (4)

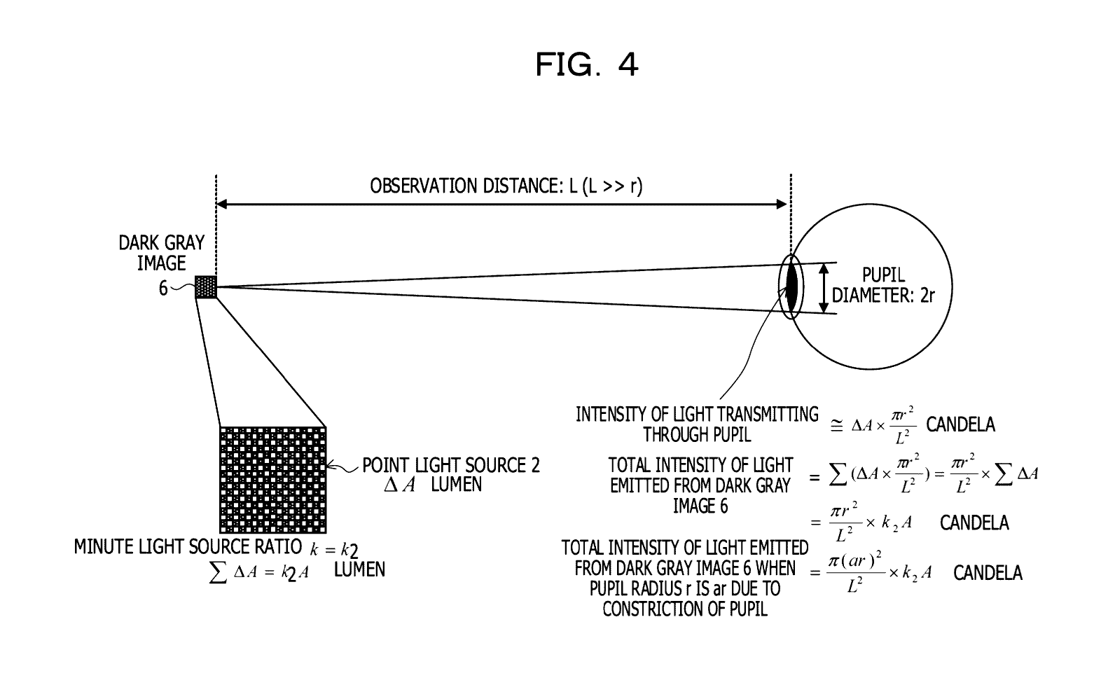

FIG. 4 is a diagram for describing a method of calculating the intensity of light arriving at a pupil from a collection of point light sources. The intensity of light arriving at a pupil from a collection of the minute point light sources 2 is calculated with reference to FIG. 4. FIG. 4 illustrates an example with the dark gray image 6. In the example illustrated in FIG. 4, Formula (3) provides the intensity of light of the dark gray image 6 when regarded as a collection of the point light sources 2 having the intensity of light at ΔA lumen.

The surface of the pupil at which the light arrives, which is spherical in reality because the eyeball is a sphere, may be regarded as a flat surface when an observation distance L from an observed image (object) to a pupil of a viewer is sufficiently larger than the radius r of the pupil of the viewer (L>>r).

Thus, the intensity of light emitted from the point light source 2 and transmitting through the pupil is given by Formula (5) based on the definition of a unit solid angle steradian.

$$the intensity of light transmitting through the pupil ≅ Δ A \times \frac{π {r}^{2}}{{L}^{2}} [ cd ] \qquad ( 5 )$$

Therefore, when the dark gray image 6 is regarded as a light source, the total intensity of light emitted from the dark gray image 6 and transmitting through the pupil is given by Formula (6).

$$the total intensity of light emitted from the dark gray image 6 = \sum ( Δ A \times \frac{π {r}^{2}}{{L}^{2}} ) = \frac{π {r}^{2}}{{L}^{2}} \times \sum Δ A = \frac{π {r}^{2}}{{L}^{2}} \times {k}_{2} A [ cd ] \qquad ( 6 )$$

The following describes a case in which the radius r of the pupil due to the constriction decreases to the radius ar (a is a value smaller than one: a<1). In this case, when the dark gray image 6 is regarded as a light source, the total intensity of light emitted from the dark gray image 6 and transmitting through the pupil is given by Formula (7) based on Formula (6).

$$the total intensity of light emitted from the dark gray image 6 = \frac{{π ( ar )}^{2}}{{L}^{2}} \times {k}_{2} A [ cd ] \qquad ( 7 )$$

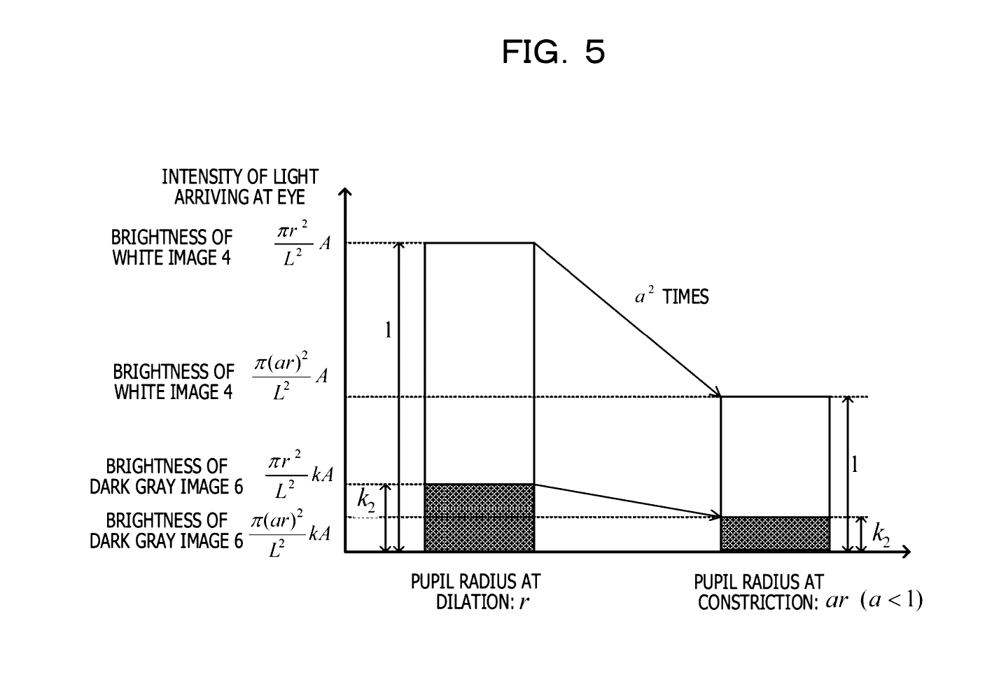

FIG. 5 illustrates a comparison of the intensity of light arriving at the retina. As illustrated in FIG. 5, when the white image 4 is regarded as a light source, the total intensity of light emitted from the white image 4 and transmitting through the pupil at the dilation (when the radius of the pupil is r) is given by Formula (8).

$$the intensity of light emitted from the white image 4 = \frac{π {r}^{2}}{{L}^{2}} \times A [ cd ] \qquad ( 8 )$$

Therefore, a ratio between the intensity of light emitted from the white image 4 and the intensity of light emitted from the dark gray image 6 at the dilation is 1:k₂ based on Formulas (6) and (8).

As illustrated in FIG. 5, when the white image 4 is regarded as a light source, the total intensity of light emitted from the white image 4 and transmitting through the pupil at constriction (when the radius of the pupil is ar) is given by Formula (9).

$$the intensity of light emitted from the white image 4 = \frac{π {( ar )}^{2}}{{L}^{2}} \times {k}_{2} A [ cd ] \qquad ( 9 )$$

Therefore, the ratio between the intensity of light emitted from the white image 4 and the intensity of light emitted from the dark gray image 6 at the constriction is 1:k₂ based on Formulas (7) and (9).

As described above, the ratio between the intensities of light emitted from images with different luminance is the same between the dilation and the constriction, and thus the relative brightness is maintained as illustrated in FIG. 5. In contrast, as illustrated in FIG. 5, the intensity of light arriving at the pupil (retina) at the constriction is a2 times larger than that at the dilation. Thus, when the constriction occurs, the intensity of light arriving at the pupil (retina) decreases in proportion to the square of the radius of the pupil, and visual recognition at a compressed contrast is obtained.

Accordingly, a motion illusion pattern is visually recognized at a decreased contrast (for example, a contrast between white and dark gray) at the constriction as compared to that at the dilation. This phenomenon is the same as a phenomenon as a symptom of presbyopia that an object is blurred due to a contrast decrease caused by the constriction of the pupils. Thus, motion illusion that has been caused by the motion illusion pattern before the constriction may disappear after the constriction in some cases when the motion illusion pattern is visually recognized by an identical person.

In the present embodiment, the above-described principle is exploited to detect the constriction of a pupil of a person (user) based on whether motion illusion that has been caused by a motion illusion pattern disappears.

The present embodiment exemplarily describes a pupil constriction detecting device configured to detect whether the constriction of the pupils due to fatigue or the like occurs to a user continuously performing visual recognition of an image displayed on a display. When what is called a head mounted display (HMD) is used, the user is likely to suffer fatigue (the constriction of the pupils).

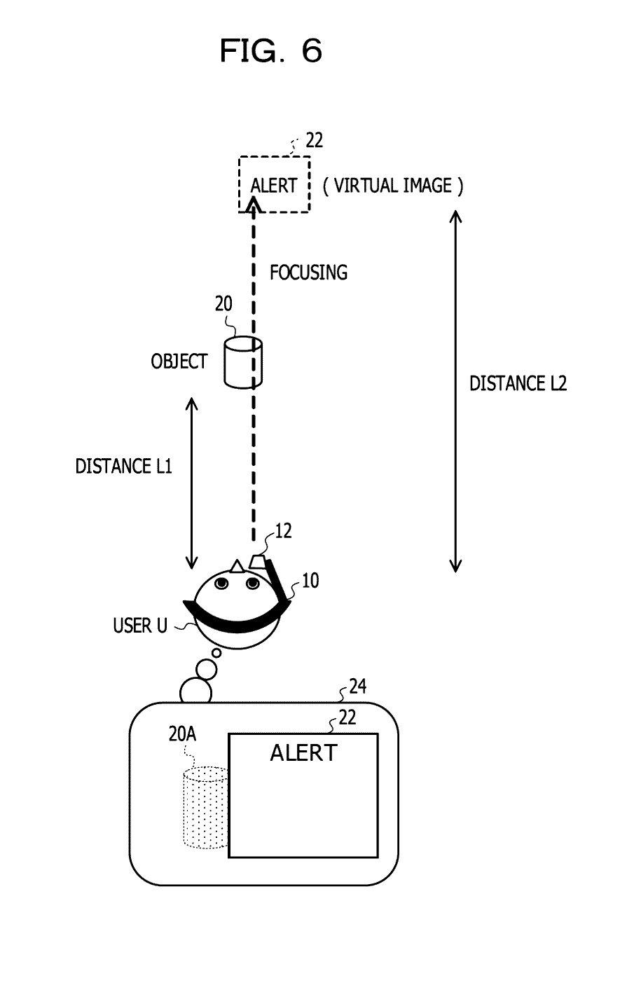

When a monocular HMD is used, typically, there is a difference between a distance to an object in the real world and a distance to a virtual image displayed on the display. The following describes an example in which, with the monocular HMD 10 mounted on the right eye as illustrated in FIG. 6, a user U visually recognizes an object 20 in the real world on which the user U performs an operation or the like and a virtual image 22.

FIG. 6 is a diagram for describing a mismatch in focusing on an object in the real world and a virtual image when a monocular HMD is used. As illustrated in FIG. 6, the object 20 is at a distance L1 the user U is capable of reaching at. A distance L2 from the user U to the virtual image 22 displayed on a display 12 of the HMD 10 is determined based on a designed value of the HMD 10, and thus it is difficult to match the distance L2 with the distance L1.

In this state, when focused on the display (the virtual image 22) of the HMD 10, the user U becomes focused off the object 20. As a result, in binocular vision, the virtual image 22 is dominant with focusing on the virtual image 22 while the object 20 is recognized as a blurred object image 20A as illustrated in a field of view 24.

Since there is a mismatch between the distance L1 to the object 20 existing in the real world and the distance L2 to the virtual image 22 as described above, the user U is likely to feel fatigue by continuously watching an unfocused image.

In addition, the user U is likely to feel fatigue due to a phenomenon called binocular rivalry when using the monocular HMD 10.

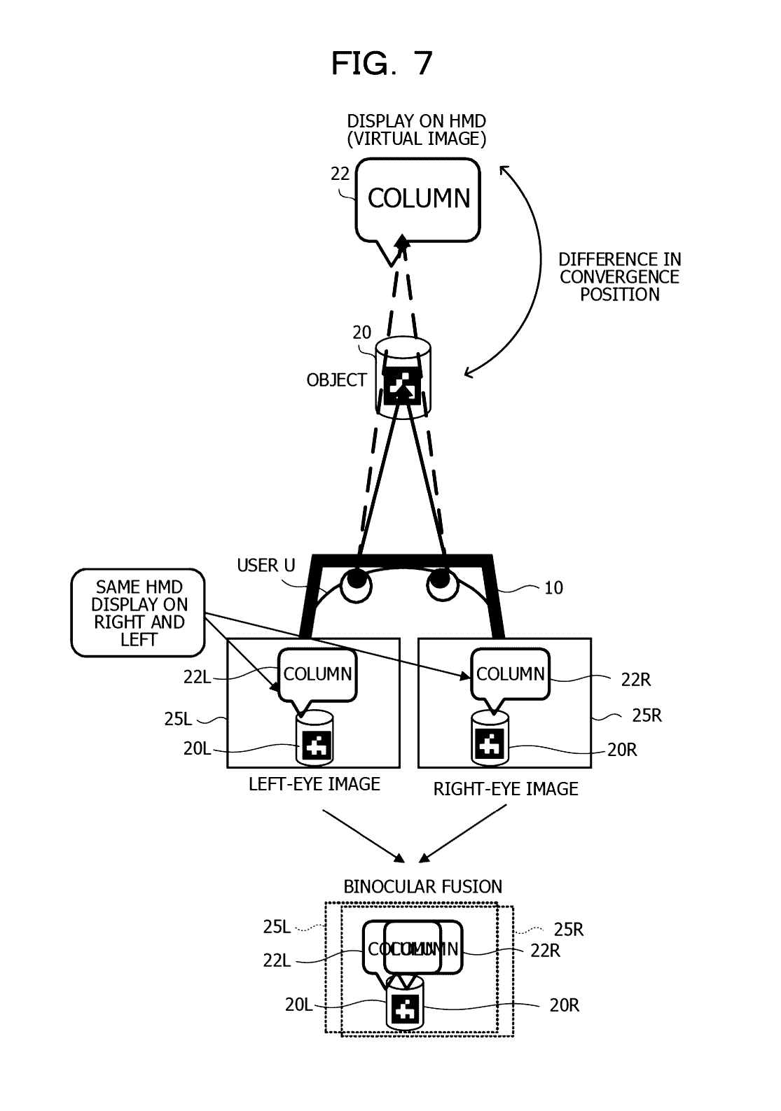

In contrast, when a binocular HMD 10 is used, typically, there is a difference between a position (fusion distance) at which an object in the real world converges and a position at which a virtual image displayed on the display converges. FIG. 7 is a diagram for describing a mismatch in the position of convergence on an object in the real world and a virtual image when the binocular HMD is used. The following describes an example in which, with the binocular HMD 10 mounted on both eyes as illustrated in FIG. 7, the user U visually recognizes the object 20 in the real world on which the user U performs an operation or the like and the display (the virtual image 22) of the HMD 10.

As illustrated in FIG. 7, there is a difference between the position of convergence on the object 20 and the position of convergence on the virtual image 22 (more specifically, a text of “column” in the virtual image 22). A left-eye image 25L visually recognized by the user U on the display 12 includes a virtual image 22L and an object image 20L. A right eye image 25R visually recognized by the user U on the display 12 includes a virtual image 22R and an object image 20R.

Thus, in binocular fusion with the left-eye image 25L and the right eye image 25R, the object image 20L and the object image 20R are appropriately visually recognized as overlapping fusion, whereas the virtual image 22L and the virtual image 22R are visually recognized as a blurred double image.

Since there is a mismatch between the position of convergence on the object 20 existing in the real world and the position of convergence on the display (virtual image 22) of the HMD 10 as described above, the user U is likely to feel fatigue by continuously watching the image with a mismatch in the position of convergence.

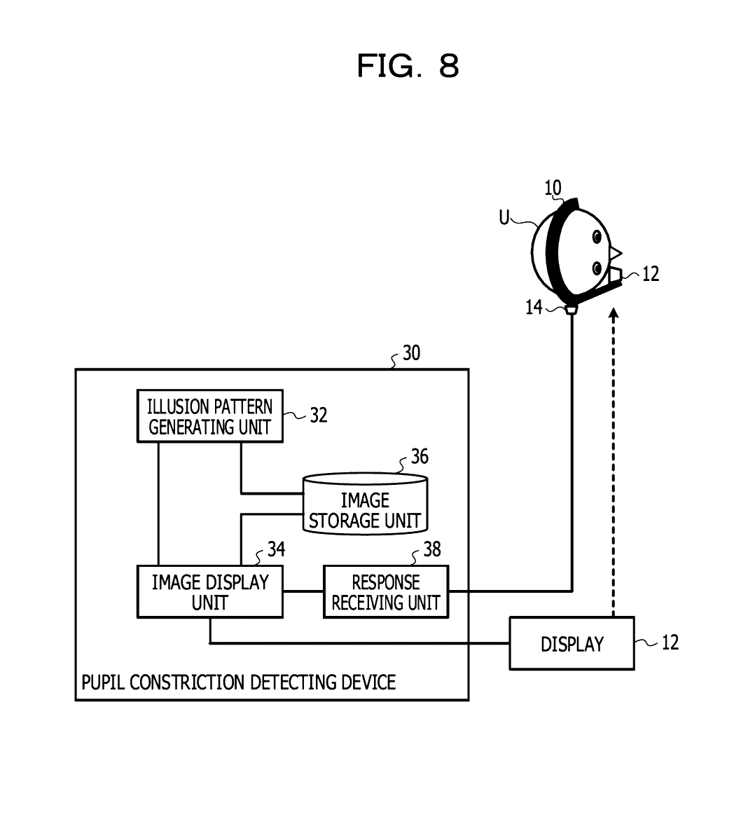

FIG. 8 illustrates an exemplary configuration of the pupil constriction detecting device according to the present embodiment. As illustrated in FIG. 8, a pupil constriction detecting device 30 according to the present embodiment includes an illusion pattern generating unit 32, an image display unit 34, an image storage unit 36, and a response receiving unit 38. As illustrated in FIG. 8, the HMD 10 according to the present embodiment is, for example, of a monocular type including the display 12 and a user response unit 14. The HMD 10 may be of what is called a see-through type or a video-through type.

The illusion pattern generating unit 32 generates a motion illusion pattern based on the instruction from the image display unit 34. The motion illusion pattern generated by the illusion pattern generating unit 32 is stored in the image storage unit 36.

For example, the illusion pattern generating unit 32 acquires image data of an original image indicating the motion illusion pattern stored in the image storage unit 36. This image data of an original image includes information defining the shape, size, and position of each image of white, light gray, dark gray, or black in the motion illusion pattern. The illusion pattern generating unit 32 generates a motion illusion pattern at a luminance value (contrast) based on the instruction from the image display unit 34 for the image data of an original image.

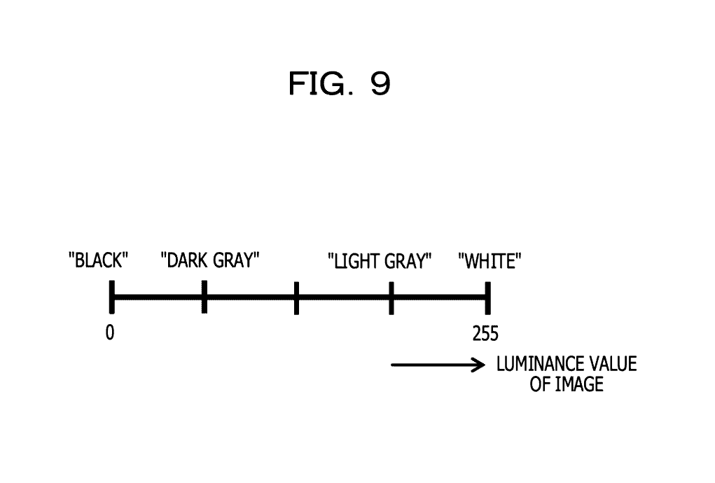

When generating the motion illusion pattern, the illusion pattern generating unit 32 according to the present embodiment performs gamma correction on the luminance value of a pixel included in the motion illusion pattern in accordance with a gamma characteristic of the display 12. FIG. 9 is a diagram for describing the luminance value in the generation of the motion illusion pattern. In the present embodiment, the luminance value indicating black, dark gray, light gray, or white in the motion illusion pattern is the 256 gradation of 0 to 255 as in a luminance distribution illustrated in FIG. 9. The luminance values of black, dark gray, light gray, and white in the original image are, for example, 0, 54, 202, and 255.

In general, the display 12 has a luminance response characteristic to an input signal, and a luminance value indicated by the input signal may be not directly proportional to the brightness of a screen. When x (with a maximum value of one) represents the luminance value and y (with a maximum value of one) represents the brightness of the screen, the characteristic of the display may be approximated by a formula y=x^{γ}. The value of γ differs depending on the model and setting of the display. Typically, when the luminance value expressed in, for example, the 256 gradation is passed to the display 12, the screen becomes darker and the motion illusion pattern displayed on the screen potentially does not cause motion illusion.

For this reason, when generating the motion illusion pattern, the illusion pattern generating unit 32 according to the present embodiment performs the gamma correction so that the brightness of the display 12 changes in a linear (directly proportional) manner as much as possible in response to a change in the luminance value.

The image display unit 34 displays the motion illusion pattern generated by the illusion pattern generating unit 32 on the display 12. The image display unit 34 also displays information for confirming, to the user U, whether motion illusion occurs to the user U, and information and the like on the fatigue of the user U. The image display unit 34 according to the present embodiment instructs the generation of the motion illusion pattern to the illusion pattern generating unit 32 based on a response from the user U received by the response receiving unit 38 (to be described in detail later).

The response receiving unit 38 receives, through the user response unit 14, the response that the user U has performed on the information and the like displayed by the image display unit 34 on the display 12. A method of the response that the user U performs on the information displayed on the image display unit 34 is not particularly limited. For example, the response may be performed by sound or by an operation on a switch or the like. The user response unit 14 and the response receiving unit 38 may each have a response receiving function in accordance with the method of the response that the user U has performed.

The image storage unit 36 stores therein the image data of the original image and the motion illusion pattern generated by the illusion pattern generating unit 32 as described above.

The pupil constriction detecting device 30 may be provided integrally with or separately from the HMD 10. When the pupil constriction detecting device 30 and the HMD 10 are separately provided, the pupil constriction detecting device 30 may be a mobile device held by the user U or a stationary device coupled with the HMD 10 through a network or the like.

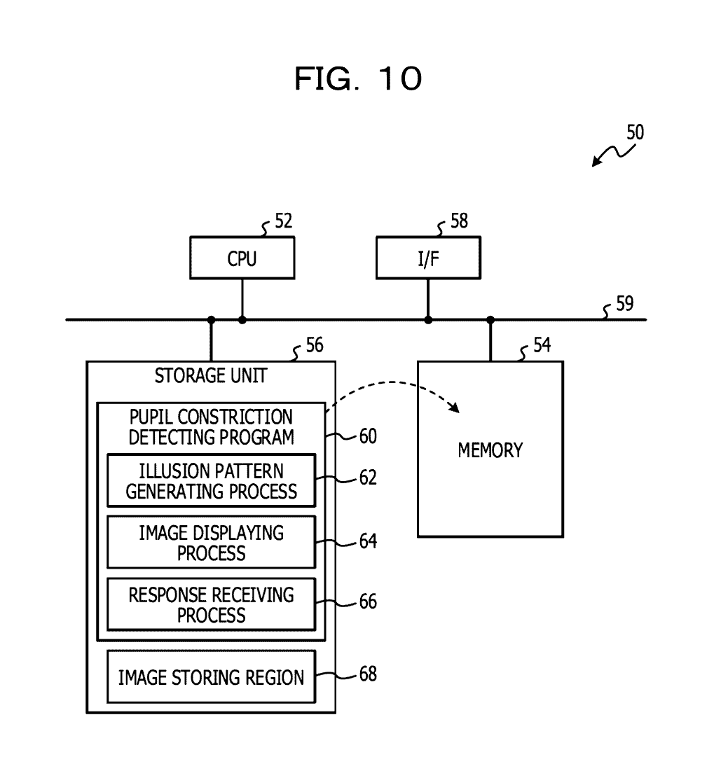

The pupil constriction detecting device 30 may be achieved by, for example, a computer 50 illustrated in FIG. 10. FIG. 10 illustrates a schematic configuration of a computer serving as the pupil constriction detecting device according to the present embodiment. For example, the pupil constriction detecting device 30 may be achieved by the computer 50 serving as a server. The computer 50 includes a central processing unit (CPU) 52, a memory 54, a non-transitory storage unit 56, and an interface (I/F) 58. The CPU 52, the memory 54, the storage unit 56, and the I/F 58 are coupled with each other through a bus 59. The I/F 58 is coupled with the display 12 and the user response unit 14 of the HMD 10.

The storage unit 56 may be achieved by a hard disk drive (HDD), a flash memory, or the like. The storage unit 56 as a storage medium stores therein a pupil constriction detecting program 60 for causing the computer 50 to function as the pupil constriction detecting device 30. The CPU 52 reads the pupil constriction detecting program 60 from the storage unit 56, loads the pupil constriction detecting program 60 onto the memory 54, and executes a process included in the pupil constriction detecting program 60.

The pupil constriction detecting program 60 includes an illusion pattern generating process 62, an image displaying process 64, and a response receiving process 66. The CPU 52 operates as the illusion pattern generating unit 32 through execution of the illusion pattern generating process 62. The CPU 52 operates as the image display unit 34 through execution of the image displaying process 64. The CPU 52 operates as the response receiving unit 38 through execution of the response receiving process 66.

Accordingly, the computer 50 executing the pupil constriction detecting program 60 serves as the pupil constriction detecting device 30. Accordingly, the storage unit 56 serves as the image storage unit 36.

The storage unit 56 includes an image storing region 68 in which, for example, a motion illusion pattern is stored.

The computer 50 is not limited to what is called a desktop personal computer. The computer 50 may be a laptop personal computer, or a personal digital assistant (PDA: mobile information terminal device) such as a tablet terminal or a smartphone.

The pupil constriction detecting device 30 may be achieved by, for example, a semiconductor integrated circuit, more specifically, an application specific integrated circuit (ASIC).

The following describes the effect of the pupil constriction detecting device 30 according to the present embodiment.

The pupil constriction detecting device 30 according to the present embodiment first performs pre-operation processing to register (store) a motion illusion pattern that causes motion illusion before the user U uses the HMD 10 (display 12).

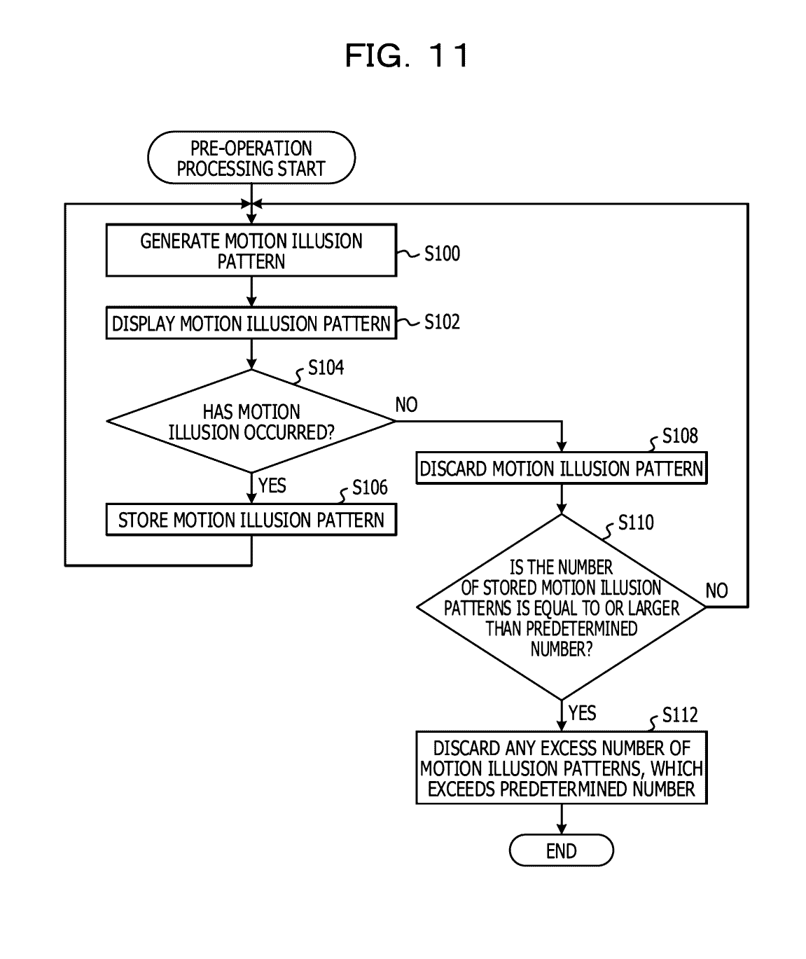

FIG. 11 is a flowchart of exemplary pre-operation processing executed at the pupil constriction detecting device according to the present embodiment. The pre-operation processing illustrated in FIG. 11 is executed, for example, when a power (not illustrated) of the HMD 10 is turned on.

At step S100, the illusion pattern generating unit 32 generates a motion illusion pattern. For example, at the start of execution of the pre-operation processing, the image display unit 34 instructs the generating of the motion illusion pattern to the illusion pattern generating unit 32. At step S100 right after the start of the pre-operation processing, the illusion pattern generating unit 32 generates the motion illusion pattern based on image data of an original image (image data with the luminance values of black, dark gray, light gray, and white at 0, 54, 202, and 255, respectively).

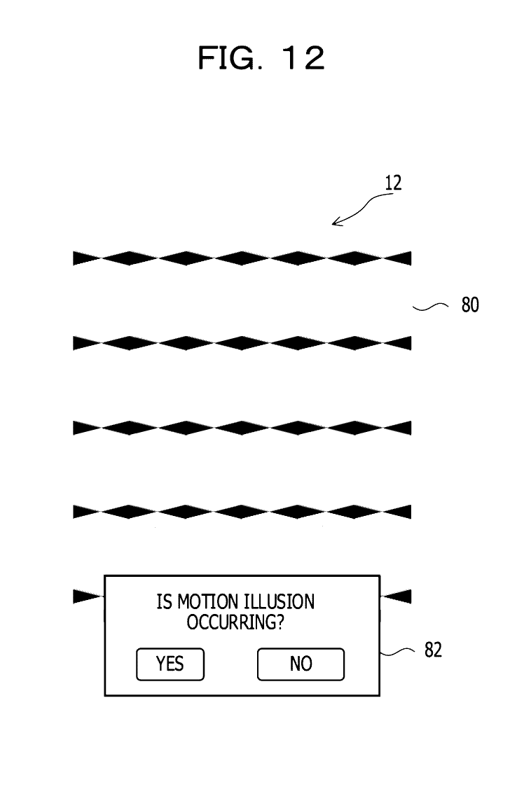

At the following step S102, the image display unit 34 displays the motion illusion pattern generated by the illusion pattern generating unit 32 on the display 12. FIG. 12 is a diagram for describing exemplary display of the motion illusion pattern on the display. As illustrated in FIG. 12, the image display unit 34 according to the present embodiment displays, on the display 12, a motion illusion pattern 80 and a message 82 prompting the user U to provide a response to tell whether motion illusion is occurring.

Upon visual recognition of the display illustrated in FIG. 12, the user U responds, through the user response unit 14, “Yes” if motion illusion is occurring or “No” if no motion illusion is occurring. The response receiving unit 38 of the pupil constriction detecting device 30 receives the response from the user U.

Then, at the following step S104, the image display unit 34 determines whether motion illusion has occurred to the user U. In the present embodiment, for example, if the user U has responded “Yes”, the image display unit 34 determines that motion illusion has occurred. In contrast, if the user U has responded “No”, the image display unit 34 determines that no motion illusion has occurred.

If it is determined that motion illusion has occurred, step S104, a positive determine is made, and then the process proceeds to step S106. At step S106, the image display unit 34 stores this motion illusion pattern 80 in the image storage unit 36, and then the process returns to step S100.

Then, again at step S100, the illusion pattern generating unit 32 generates the motion illusion pattern 80. In the present embodiment, for example, as illustrated in FIG. 12, the motion illusion pattern 80 with highest luminance values of black, dark gray, light gray, and white is used as an original image. Thus, the motion illusion pattern 80 newly generated with different luminance values has an entire luminance value (contrast) lower than that of the motion illusion pattern 80 previously generated.

For example, the illusion pattern generating unit 32 allocates, to each color, a luminance value obtained by multiplying the luminance value of a previously generated motion illusion pattern by the square of a predetermined attenuation coefficient.

As described above, the luminance values of (black, dark gray, light gray, white) of the motion illusion pattern 80 generated for the first time are (0, 54, 202, 255).

For example, when the attenuation coefficient at the second time generation is 0.9, the luminance values of (black, dark gray, light gray, white) of the motion illusion pattern 80 generated for the second time are (0, 44, 164, 207). When the attenuation coefficient at the third time generation is 0.8, the luminance values of (black, dark gray, light gray, white) of the motion illusion pattern 80 generated for the third time are (0, 35, 129, 163). When the attenuation coefficient at the fourth time generation is 0.7, the luminance values of (black, dark gray, light gray, white) of the motion illusion pattern 80 generated for the fourth time are (0, 26, 99, 125). The luminance values are rounded to the nearest integers in the calculation.

At repetition of the generation of the motion illusion pattern 80, the illusion pattern generating unit 32 according to the present embodiment reduces the luminance values (contrasts) of the motion illusion pattern 80 thus generated by gradually reducing the attenuation coefficient as described above.

The pupil constriction detecting device 30 according to the present embodiment sequentially generates and displays the motion illusion pattern 80 at compressed contrasts by entirely decreasing the luminance values through multiplication by the square of the attenuation coefficient as described above. This enables display of the motion illusion pattern 80 optimum for detecting the constriction of the pupils of the user U, in other words, the motion illusion pattern 80 that causes motion illusion to the user U and has a relatively low entire luminance (contrast).

The attenuation coefficient is not limited to that in the present embodiment, but may be determined depending on, for example, the kind of the motion illusion pattern.

If the image display unit 34 determines that no motion illusion is visually recognized at the above-described step S104, the process proceeds to step S108.

At step S108, the illusion pattern generating unit 32 discards the motion illusion pattern 80 displayed on the display 12 (discards image data of the motion illusion pattern 80), and then the process proceeds to step S110.

At step S110, the image display unit 34 determines whether the number of motion illusion patterns 80 stored in the image storage unit 36 is equal to or larger than a predetermined number. If the number of stored motion illusion patterns 80 is not equal to or larger than the predetermined number, a negative determination is made, and the process returns to step S100.

Whether an identical motion illusion pattern 80 causes motion illusion depends on individual persons as described above. Thus, if no motion illusion occurs to the user U for the number of motion illusion patterns 80 equal to or larger than the predetermined number, the motion illusion pattern 80 of a different kind is generated. In the present embodiment, motion illusion patterns of different kinds include a motion illusion pattern in which at least one of the shape, size, dispose, and color of an image of each color is different.

The “kind” is a difference in the pattern of arrangement of black, dark gray, light gray, and white or a difference in color of black, dark gray, light gray, and white.

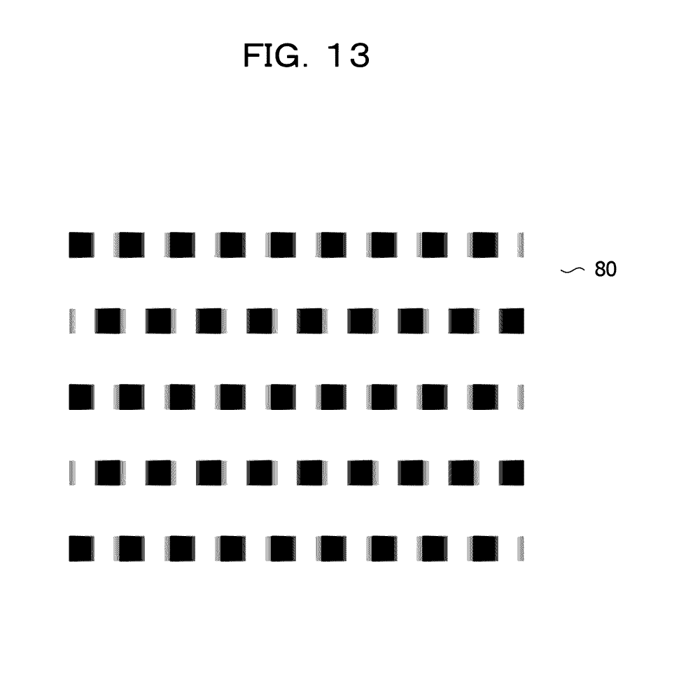

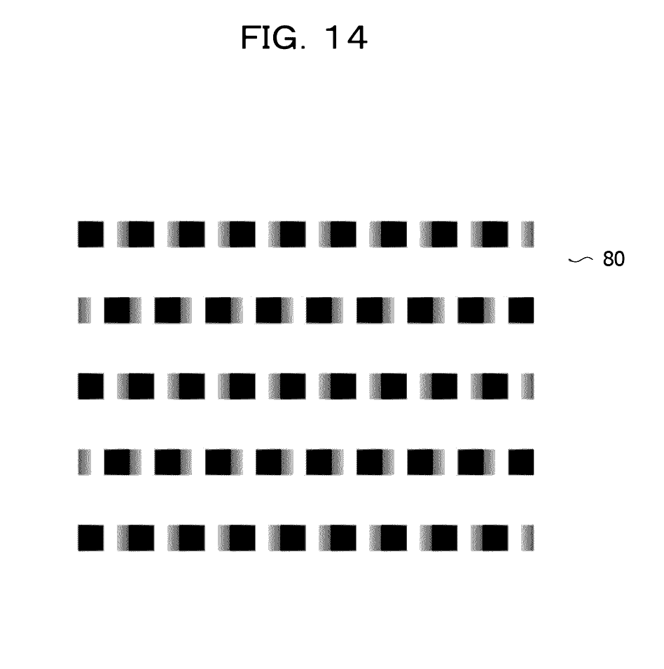

FIG. 13 illustrates an exemplary motion illusion pattern 80 with different patterns from the motion illusion pattern 80 illustrated in FIGS. 1 and 2. FIG. 14 illustrates a motion illusion pattern 80 with an entire luminance 20% lower than that of the exemplary motion illusion pattern 80 illustrated in FIG. 13. The motion illusion pattern illustrated in FIGS. 1 and 2 includes a combination of triangular images, whereas the motion illusion pattern 80 illustrated in FIGS. 13 and 14 includes a combination of rectangular images.

In the motion illusion pattern 80, the luminance within an individual image is not limited to a fixed value, but may have a luminance gradient within the image. In the example illustrated in FIG. 13, similarly to the motion illusion pattern in FIG. 1, the luminance values of black, dark gray, light gray, and white are 0, 54, 202, and 255, respectively. The luminance is linearly interpolated to have a luminance gradient in a region between the position of black (luminance value=0) and the position of dark gray (luminance value=54). The luminance is also extrapolated with an equation of the interpolation to have a luminance gradient in a region between the position of dark gray (luminance value=54) and the position of white (luminance value=255). The luminance is also linearly interpolated to have a luminance gradient in a region between the position of white (luminance value=255) and the position of light gray (luminance value=202). The luminance is also extrapolated with an equation of the interpolation to have a luminance gradient in a region between the position of light gray (luminance value=54) and the position of black (luminance value=0). As a result, a boundary between black and dark gray and a boundary between white and light gray do not have to be visually recognized.

The “interpolation” in the present embodiment calculates, based on a known numerical data array, a numerical value filling the range of an interval of the data array. The “extrapolation” calculates, based on a known numerical data, a numerical value expected outside the range of the data.

The motion illusion pattern 80 illustrated in FIGS. 13 and 14 causes motion illusion that appears to be moving in a direction in which black, dark gray, light gray, and white are arranged.

When the motion illusion pattern 80 is generated with colors different from black, dark gray, light gray, and white, a boundary of each color (image) has to be formed not based on the kinds of colors but based on a luminance difference while the above-described condition is satisfied. A wavelength at which a rod cell governing peripheral vision has a maximum sensitivity is 505 nm, which corresponds to (dark) green. Thus, formula of the brightness by color exploits a fact that a dark color may be expressed by a mixed color of red and blue, which are distant from green, and a light color may be expressed by a color (for example, yellow) closer to green. For example, purple at (R, G, B)=(0x78, 0x00, 0xA0) may be used in place of dark gray. For example, yellow at (R, G, B)=(0xFF, 0x0FF, 0x00) may be used in place of white, which is brightest. For example, yellow-green at (R, G, B)=(0xAE, 0x0ff, 0x00) may be used in place of light gray.

For example, in order to have a different color variation in the motion illusion pattern 80 illustrated in FIG. 13, (R, G, B)=(0x78, 0x00, 0x78) may be used in place of dark gray. Yellow (R, G, B)=(0xFF, 0xFF, 0x00) may be used in place of white, which is brightest. (R, G, B)=(0xD2, 0xD2, 0x00) may be used in place of light gray.

If the number of stored motion illusion patterns 80 is equal to or larger than the predetermined number at step S110, a positive determine is made, and then the process proceeds to step S112. The predetermined number may be any number equal to or larger than one, but is preferably equal to or larger than two.

At step S112, the image display unit 34 discards any excess number of motion illusion patterns 80 stored in the image storage unit 36, which exceeds the predetermined number, and then the pre-operation processing ends. It is preferable not to discard but to store, in the image storage unit 36, any motion illusion pattern 80 having luminance values within a predetermined range from the luminance values of the motion illusion pattern 80 with which motion illusion occurring to the user U disappears. For example, the motion illusion pattern 80 displayed on the display 12 right before motion illusion occurring to the user U disappears is preferably stored in the image storage unit 36. Moreover, it is preferable to set the predetermined number to be equal to or larger than two to store the motion illusion patterns 80 including the motion illusion pattern 80 with luminance values at which a motion illusion has clearly occurred to the user U and the motion illusion pattern 80 displayed right before motion illusion disappears.

Storing a plurality of the motion illusion patterns 80 enables more precise determination of whether the constriction of the pupils occurs to the user U in pupil constriction detecting processing to be described later in detail.

Alternatively, in the pre-operation processing described above, each user U may be recognized to store, for the user U, the kinds and luminance values of the motion illusion pattern 80 stored in the image storage unit 36 through the pre-operation processing. This information is used to generate the motion illusion pattern 80 at the next execution of the pre-operation processing. For example, the initial values of luminance of the motion illusion pattern 80 displayed to the user U are set to the luminance values of the motion illusion pattern 80 used in the past in place of maximum luminance values. This allows a time taken for the pre-operation processing to be shortened.

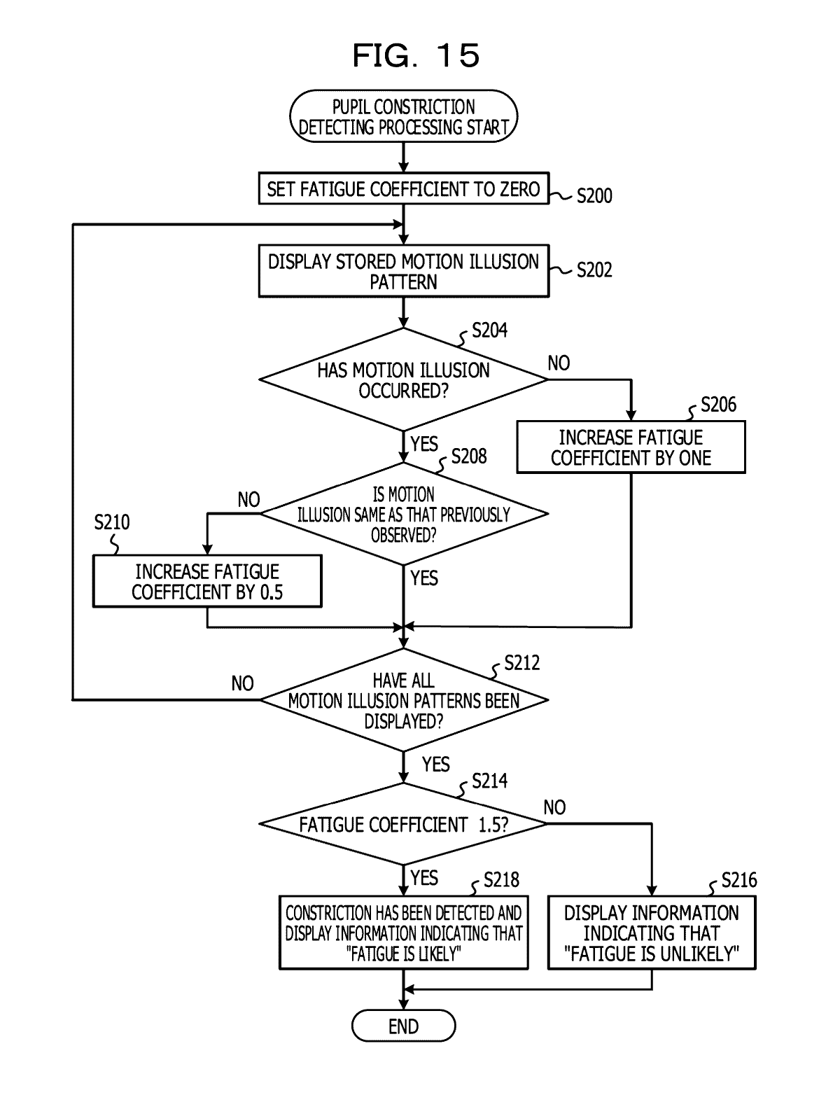

After the pre-operation processing ends in this manner, the user U performs an operation or the like using the HMD 10, and visually recognizes an image displayed on the display 12. After a predetermined time has passed from the start of the operation by the user U, for example, after a predetermined time has passed from the end of the pre-operation processing, the pupil constriction detecting device 30 according to the present embodiment executes the pupil constriction detecting processing illustrated in FIG. 15. The predetermined time is not particularly limited, but may be determined depending on a typical time experimentally obtained in advance in which the user U has the feeling of fatigue from the use of the HMD 10. The predetermined time is, for example, a time in a range of 5 minutes to 20 minutes. The predetermined time may be switched for each user U based on past records. FIG. 15 is a flowchart of exemplary pupil constriction detecting processing executed at the pupil constriction detecting device according to the present embodiment.

At step S200, the image display unit 34 sets a fatigue coefficient to zero.

At the following step S202, the image display unit 34 displays the motion illusion pattern 80 stored in the image storage unit 36 on the display 12. If a plurality of the motion illusion patterns 80 are stored in the image storage unit 36, the motion illusion patterns 80 are displayed sequentially (one by one) on the display 12. In this case, the order of displaying of the motion illusion patterns 80 is not particularly limited, and the motion illusion patterns 80 may be displayed, for example, in the descending order of the luminance values (contrasts). The display method is not particularly limited and may be performed, for example, as illustrated in FIG. 12 similarly to step S102 of the pre-operation processing (refer to FIG. 11).

In response to this display, the user U provides, through the user response unit 14, a response on whether motion illusion is occurring. Then, the response receiving unit 38 of the pupil constriction detecting device 30 receives this response from the user U.

At the following step S204, the image display unit 34 determines whether the user U has visually recognized motion illusion, similarly to step S104 of the pre-operation processing (refer to FIG. 11). If the user U has not visually recognized motion illusion, a negative determination is made, and then the process proceeds to step S206.

At step S206, the image display unit 34 increases the fatigue coefficient by one, and then the process proceeds to step S212.

If the user U has visually recognized motion illusion at step S204, a positive determination is made, and then the process proceeds to step S208.

At step S208, the image display unit 34 determines whether the motion illusion occurring to the user U is the same as that previously observed. This determination may be performed, for example, based on a response received from the user U by the response receiving unit 38 through the user response unit 14 to the message 82 in FIG. 12 displayed on the display 12. For example, when the constriction of the pupils is occurring, motion illusion is occurring but is lower (has less motion) than previously observed. In such a case, the user U provides a response that motion illusion is not the same as that previously observed.

If motion illusion is not the same as that previously observed, a negative determination is made, and then the process proceeds to step S210. At step S210, the image display unit 34 increases the fatigue coefficient by 0.5, and then the process proceeds to step S212.

If motion illusion is the same as that previously observed, a positive determine is made, and then the process proceeds to step S212. At step S212, the image display unit 34 determines whether all the motion illusion patterns 80 stored in the image storage unit 36 have been displayed. If there is the motion illusion pattern 80 yet to be displayed, the process returns to step S202 and repeats the above-described processing.

If all the motion illusion patterns 80 have been displayed, a positive determine is made, and then the process proceeds to step S214.

At step S214, the image display unit 34 determines whether the fatigue coefficient is equal to or larger than 1.5 (the fatigue coefficient≧1.5). If the fatigue coefficient is smaller than 1.5, a negative determination is made, and then the process proceeds to step S216. At step S216, the image display unit 34 displays information indicating that “fatigue is unlikely” on the display 12, and then the pupil constriction detecting processing ends.

If the fatigue coefficient is equal to or larger than 1.5, a positive determine is made, and then the process proceeds to step S218. At step S218, the image display unit 34 determines that the constriction of the pupils of the user U has been detected, and displays information indicating that “fatigue is likely” on the display 12, and then the pupil constriction detecting processing ends.

The pupil constriction detecting processing is preferably repeated each time the predetermined time has passed while the user U is continuously using the HMD 10. When the pupil constriction detecting processing is repeated, the user U is more likely to have fatigue as a passed operation time increases, and thus an interval at which the pupil constriction detecting processing is performed may be set short (for example, gradually shorter as the operation time passes).

The information indicating a likelihood that the user U has fatigue at steps S216 and S218 of the pupil constriction detecting processing may be externally output from the pupil constriction detecting device 30. Externally outputting the information indicating the likelihood that the user U has fatigue in this manner allows the fatigue of the user U to be externally monitored. The information indicating the likelihood that the user U has fatigue may be stored in, for example, the image storage unit 36 in association with a time in which the pupil constriction detecting processing is executed, an elapsed time from the start of the operation, or the like.

In the pupil constriction detecting processing, the fatigue coefficient of “1” is applied if motion illusion having been caused to the user U by the motion illusion pattern 80 before the operation disappears, or the fatigue coefficient of “0.5” is applied if motion illusion is less significant. However, the fatigue coefficient to be applied in these cases is not limited to those coefficients. In the pre-operation processing, the fatigue coefficient may differ between the motion illusion pattern 80 displayed on the display 12 right before motion illusion occurring to the user U disappears, and the other motion illusion patterns 80. Alternatively, the fatigue coefficient may differ depending on the luminance values (contrasts) of the motion illusion pattern 80 or depending on the kind of the motion illusion pattern 80 or the like.

In the pupil constriction detecting processing, the constriction of the pupils is detectable when the fatigue coefficient is equal to or larger than 1.5, and thus it is determined that the user U is likely to have fatigue. However, the fatigue coefficient as a reference for detecting the constriction of the pupils is not limited to “1.5”. For example, the fatigue coefficient as the reference for detecting the constriction of the pupils may be an experimentally obtained value when the user U is using the HMD 10. Alternatively, the fatigue coefficient may be switched for each user U based on past records.

In the pupil constriction detecting processing, it is detected based on the fatigue coefficient whether the constriction of the pupils is occurring to the user U, in other words, whether the user U has fatigue, but the fatigue coefficient does not have to be used. For example, the constriction of the pupils of the user U may be detected to determine that the user U has fatigue if motion illusion occurred to the user U by the motion illusion pattern 80 before the operation disappears.

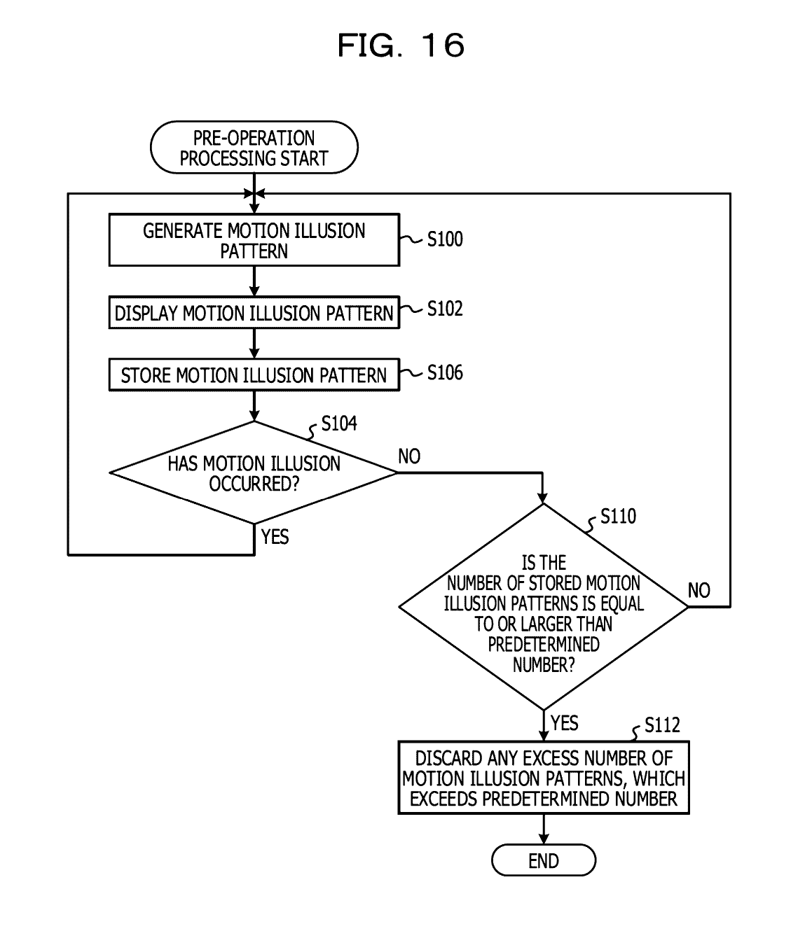

In the pre-operation processing, the motion illusion pattern 80 causing no motion illusion to the user U is discarded without being stored in the image storage unit 36, but may be stored in the image storage unit 36. In this case, in the pre-operation processing, steps S104 and S106 of the pre-operation processing (refer to FIG. 11) are performed in the opposite order and the processing at step S108 may be omitted as illustrated in FIG. 16. In this case, the motion illusion pattern 80 causing no motion illusion to the user U is displayed in the pupil constriction detecting processing. As described above, in the pre-operation processing, the pupil constriction detecting processing is performed using the motion illusion pattern 80 causing no motion illusion to the user U, which enables more precise determination of whether the constriction of the pupils is occurring to the user U.

Alternatively, only the motion illusion pattern 80 with a lowest luminance among the motion illusion patterns 80 occurring motion illusions to the user U may be stored in the image storage unit 36.

In the present embodiment, although the pre-operation processing is performed when the user U starts the operation with the HMD 10, the timing of the pre-operation processing is not limited thereto. For example, the pre-operation processing may be performed in advance irrespective of a timing when the user U performs the operation, and the motion illusion pattern 80 may be stored in the image storage unit 36 in association with identification information for identifying the user U. In this case, when the user U uses the HMD 10, identification of the user U may be performed based on the identification information received by the response receiving unit 38 through, for example, the user response unit 14, and the pupil constriction detecting processing may be performed using the motion illusion pattern 80 in accordance with the identified user U. The pre-operation processing may be performed only once for each individual user U, and only the pupil constriction detecting processing may be performed in the following execution.

In the present embodiment, the motion illusion pattern 80 generated by the illusion pattern generating unit 32 is stored in the image storage unit 36 in the pre-operation processing. However, the luminance values of the motion illusion pattern 80 may be stored in place of the motion illusion pattern 80. This configuration includes additional processing of generating the motion illusion pattern 80 based on the luminance values stored in the pupil constriction detecting processing, but downsizing of the capacity of the image storage unit 36 is achieved.

The present embodiment describes the case in which the pupil constriction detecting device 30 detects the constriction of the pupils of the user U when using the display 12 of the HMD 10, but a condition that the constriction of the pupils of the user U is detected is not limited thereto. For example, the pupil constriction detecting device 30 may be applied to detection of the constriction of the pupils of the user U under a condition that the user U performs a VDT operation using a display device such as a normal monitor.

As described above, the pupil constriction detecting device 30 according to the present embodiment stores in advance the motion illusion pattern 80 with luminance values causing motion illusion to the user U in the image storage unit 36. The image display unit 34 displays the motion illusion pattern 80 stored in the image storage unit 36 on the display 12 to allow determination of whether motion illusion occurs to the user U through visual recognition of an image displayed on the display 12.

Therefore, the pupil constriction detecting device 30 according to the present embodiment is capable of detecting the constriction of the pupils of the user with a simple configuration without using hardware such as an image capturing apparatus configured to capture an image of the eyes of the user U.

The pupil constriction detecting device 30 according to the present embodiment allows the user U to recognize the fatigue thereof. Accordingly, the user U is allowed to take a rest appropriately depending on the fatigue thereof.

In the above description, the pupil constriction detecting program 60 is stored (installed) in the storage unit 56 of the computer 50 in advance. However, the pupil constriction detecting program 60 may be recorded in a storage medium and provided. The storage medium is, for example, a Compact Disc Read Only Memory (CD-ROM), a Digital Versatile Disk Read Only Memory (DVD-ROM), or a Universal Serial Bus (USB) memory.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiment of the present invention has been described in detail, it should be understood that the various changes, substitutions, and alterations could be made hereto without departing from the spirit and scope of the invention.

What is claimed is:

1. A method of detecting constriction of the pupils, the method comprising:
registering one or more motion illusion pattern images or one or more luminance values of the one or more motion illusion pattern images, the one or more motion illusion pattern images including a first motion illusion pattern image which had caused motion illusion to a specific user;
first displaying the registered one or more motion illusion pattern images or one or more motion illusion pattern images created based on the registered one or more luminance values on a display device; and
first determining, by a processor, whether the motion illusion is occurred to the specific user based on the first displaying.

2. The method according to claim 1, wherein
the first determining includes:
receiving, from the specific user, a result of confirmation of whether the motion illusion pattern is caused motion illusion to the specific user, and
the method further comprises:
detecting constriction of the pupils of the specific user based on the result of confirmation.

3. The method according to claim 2, wherein
the receiving receives the result of confirmation for each of the registered one or more motion illusion pattern images or one or more motion illusion pattern images created based on the registered one or more luminance values displayed by the first displaying,
the first determining includes:
first increasing, for each of receiving the result of confirmation, a fatigue coefficient by a first value when the result of confirmation indicates that the motion illusion is not occurred to the specific user, and
the detecting detects the constriction of the pupils of the specific user based on a value of the fatigue coefficient when receiving the result of confirmation with regard to all of the registered one or more motion illusion pattern images or one or more motion illusion pattern images created based on the registered one or more luminance values.

4. The method according to claim 3, wherein
the first determining includes:
second increasing, for each of receiving the result of confirmation, the fatigue coefficient by a second value when the result of confirmation indicates that the motion illusion other than a motion illusion had occurred to the specific user in the past is occurred to the specific user.

5. The method according to claim 1, wherein the display device is a head mounted display device (HMD).

6. The method according to claim 5, further comprising:
repeatedly executing the first displaying and the first determining during the specific user uses the HMD.

7. The method according to claim 6, wherein
according to a length of elapsed time when the specific user uses the HMD during the specific user uses the HMD, a time interval from a previous executing to a subsequent executing in the repeatedly executing is gradually shortened.

8. The method according to claim 1, wherein
the registering stores the one or more motion illusion pattern images or the one or more luminance values in association with identification information for identifying a user into a memory,
the method further comprises:
second receiving identification information indicating the specific user inputted when the specific user uses the HMD,
the first displaying displays the registered one or more motion illusion pattern images or the one or more motion illusion pattern images created based on the registered one or more luminance values in the memory corresponding to the received identification information on the HMD.

9. The method according to claim 1, wherein
the registering includes:
second displaying a plurality of motion illusion pattern images having luminance value differ from each other on the display device, and
second determining whether a motion illusion is occurred to the specific user for each of plurality of motion illusion pattern images based on the second displaying.

10. The method according to claim 9, wherein
the first motion illusion pattern is determined by the second determining that the motion illusion has occurred to the specific user, and has a first luminance value within a predetermined value range from a luminance value of a motion illusion pattern image determined by the second determining that no motion illusion has occurred to the specific user.

11. The method according to claim 10, wherein
the one or more motion illusion pattern images include a second motion illusion pattern image determined by the second determining that the motion illusion has occurred to the specific user, the second motion illusion pattern image having a second luminance value,
the first luminance value and the second luminance value have a relation capable of being approximated by a quadratic curve.

12. The method according to claim 9, wherein
the one or more motion illusion pattern images include a third motion illusion pattern image determined by the second determining that no motion illusion has occurred to the specific user.

13. The method according to claim 9, wherein
the registering registers a motion illusion pattern image having a luminance value which is the lowest among one or more motion illusion pattern images or the luminance value, the one or more motion illusion pattern images are determined by the second determining that the motion illusion has occurred to the specific user among the plurality of motion illusion pattern images.

14. The method according to claim 9, wherein
the registering includes:
preliminary registering, among the plurality of motion illusion pattern images, one or more motion illusion pattern images determined by the second determining that the motion illusion has occurred to the specific user, and
when it is determined that a number of the preliminary registered one or more motion illusion pattern images is greater than a predetermined number, deleting one or more of the preliminary registered one or more motion illusion pattern images exceeding the predetermined number.

15. The method according to claim 9, wherein
the registering includes:
third displaying another motion illusion pattern images other than the plurality of motion illusion pattern images when a number of motion illusion pattern images determined by the second determining that the motion illusion has occurred to the specific user is less than a predetermined number, each of the another motion illusion pattern images being different from each of the plurality of motion illusion pattern images with regard to at least one of shape, size, position, and color of an image of each color included in the motion illusion pattern images, and
second determining whether the motion illusion is occurred to the specific user for each of the another motion illusion pattern images based on the third displaying.

16. The method according to claim 9, further comprising:
creating the plurality of motion illusion pattern images by performing gamma correction so that a brightness of the display device changes in a liner manner in response to a change in a luminance value of each pixel included in the motion illusion pattern image for each of the plurality of motion illusion pattern images.

17. The method according to claim 9, wherein
a luminance value of a fourth motion illusion pattern image included in the plurality of motion illusion pattern images is based on a value obtained by multiplying a luminance value of a fifth motion illusion pattern image included in the plurality of motion illusion pattern images by a square of a predetermined attenuation coefficient.

18. An apparatus comprising:
a memory configured to store one or more motion illusion pattern images or one or more luminance values, the one or more motion illusion pattern images including a motion illusion pattern image which had caused motion illusion to a specific user, the one or more luminance values including a luminance value of a motion illusion pattern image which had caused motion illusion to the specific user; and
a processor coupled to the memory and configured to:
display the one or more motion illusion pattern images in the memory or one or more motion illusion pattern images created based on the one or more luminance values in the memory on a display device, and
determine whether the motion illusion is occurred to the specific user based on the displayed one or more motion illusion pattern images.

19. A non-transitory storage medium storing a program for causing a computer including a memory to execute a process, the process comprising:
reading one or more motion illusion pattern images or one or more luminance values from the memory, the one or more motion illusion pattern images including a motion illusion pattern image which had caused motion illusion to a specific user, the one or more luminance values including a luminance value of a motion illusion pattern image which had caused motion illusion to the specific user;
displaying the read one or more motion illusion pattern images or one or more motion illusion pattern images created based on the read one or more luminance values on a display device; and
determining whether the motion illusion is occurred to the specific user based on the displayed one or more motion illusion pattern images.
